(19) United States
(12) Patent Application Publication
(10) Pub. No.: US 2026/0319114 A1
(43) Pub. Date: Oct. 15, 2026

(54) DATA CORRECTION METHOD, DATA CORRECTION METHOD FOR A BAD CHANNEL OF A MULTI-SOURCE CT SYSTEM, AND MULTI-SOURCE COMPUTED TOMOGRAPHY SYSTEM

(71) Applicant: BEIJING PHOTON COUNTING TECHNOLOGY LTD., Beijing (CN)

(72) Inventors: Zhanfeng XING, Beijing (CN); Miao YU, Beijing (CN); Xin WANG, Beijing (CN); Weiqiang ZHAO, Beijing (CN); Na GAO, Beijing (CN); Wanfeng LI, Beijing (CN); Hao XU, Beijing (CN)

(21) Appl. No.: 19/482,101

(22) PCT Filed: Dec. 20, 2024
(86) PCT No.: PCT/CN2024/140877

§ 371 Date: Nov. 6, 2025

(30) Foreign Application Priority Data
Oct. 31, 2023 (CN) 202311286387.7

(51) Int. Cl. G01T 1/29; G06T 12/10

(52) U.S. Cl. CPC G01T 1/2985; G06T 12/10

(57) ABSTRACT

A data correction method, data correction method for a bad channel of a multi-source CT system, and a multi-source computed tomography system. The data correction method includes: determining a position and distribution of a bad channel in a first projection of a first combination of tube and detectors; determining an opposite projection data, which corresponds to a channel opposite to the bad channel, in a second projection of a second combination of tube and detectors, in a case where the distribution of the bad channel does not meet a preset condition; and correcting a projection data of the bad channel by using the opposite projection data.

### CROSS-REFERENCE TO RELATED APPLICATIONS

The present disclosure is based on and claims the priority to the Chinese application No. 202311286387.7 filed on Oct. 31, 2023, the disclosure of which is hereby incorporated in its entirety into the present disclosure.

### TECHNICAL FIELD

The present disclosure pertains to the application field of X-ray medical imaging equipment, and specifically is a data correction method, a data correction method for a bad channel of a multi-source CT system, and a multi-source computed tomography system.

### BACKGROUND

In a CT (Computed Tomography) system, in order to obtain a reconstructed image with good quality, there is a need to pre-process acquired projection data, which relates to determining the accuracy and reliability of signal reading from CT detectors, and also includes whether the performance of each channel (also referred to as a pixel) itself of the detectors is normal. If the data is abnormal and the quantity is within a certain range, there is a need to correct data from an abnormal channel of the detectors. If a module containing the bad channel is completely discarded, it will result in a significant decrease in the yield of detectors, and a significant increase in costs.

With unit size becoming smaller and smaller and number of rows of the CT detectors becoming larger and larger, integration level of the detectors and a data acquisition system is becoming higher and higher, resulting in more and more occurrences of bad channels in the detectors.

In a photon-counting CT system, in addition to the above factors, imperfections and defects in a direct conversion semiconductor material for detectors also result in a significant increase in a proportion of bad channels, compared to proportion of bad channels in detectors of indirect conversion.

In a conventional single-source CT system, for detection of a bad channel, determination can be made by an average response of detectors module (CN112656435A), and correction is generally performed by replacing data of the bad channel with a data difference of adjacent channels (CN104939859A) or by rotating projection data of another channel by 180 degrees (CN201410428793.7).

However, since for an area closer to a rotation center (central channel, the channel whose ray passes the isocenter, which is the rotation center of the gantry), a source of its reconstruction data is more homogeneous, and the data required for reconstruction is almost completely from the central channel. Moreover, a structure inside a human body is relatively complex, and inside a scanned area, changes are not uniform or slow, and therefore, for the detectors data close to the central channel, correction by a difference is prone to artifacts.

Therefore, in the conventional CT system, there are relatively strict requirements for a distribution of the bad channels. For example: no adjacent bad channels; no bad channels within a view range of 16 cm; and no bad channels at an outermost layer of the scan, etc. Data correction for the bad channel is thus limited by the above conditions.

### SUMMARY

In order to solve the defects and shortcomings in the related art, the present disclosure provides data correction method for a bad channel of a multi-source CT system, for ensuring the image quality, improving the yield of detectors, and reducing the system costs.

According to an aspect of the present disclosure, there is provided A data correction method for a multi-source computed tomography (CT) system, comprising: determining a position and distribution of a bad channel in a first projection of a first combination of tube and detectors; determining an opposite projection data, which corresponds to a channel opposite to the bad channel, in a second projection of a second combination of tube and detectors, in a case where the distribution of the bad channel does not meet a preset condition; and correcting a projection data of the bad channel by using the opposite projection data.

According to another aspect of the present disclosure, there is provided a data correction method for a bad channel of a multi-source CT system, comprising:
step 1. determining a position and distribution of a bad channel by a linearity check, through acquiring data of different tube currents in the system for multiple times;
step 2. interpolating projection data of adjacent channels of the bad channel in a current detectors, to correct the projection data of the bad channel, in a case where the distribution of the bad channel meets conditions of:
a) adjacent channels each being a good channel;
b) the bad channel being not located within a certain view range close to a central channel; and
c) the bad channel being not located at an outermost row in a multi-detector acquisition mode supported by the CT system;
step 3. checking whether a previous combination of tube and detectors or next combination of tube and detectors is capable of providing opposite projection data to correct the data of the target bad channel, in a case where the distribution of the bad channel does not meet the conditions in step 2;
step 4. selecting the opposite projection data according to the number of combinations of tube and detectors of the CT system and relative position between combinations of tube and detectors, and then correcting the data, and entering a subsequent pre-processing process, in a case where it is capable to correct the target bad channel by using the opposite projection data of the previous combination of tube and detectors or the next combination of tube and detectors; and
step 5. calculating a corresponding channel according to the opening angle between the corresponding channel and the central channel, after determining the corresponding combination of tube and detectors and sampling angle, wherein a corresponding projection value can be calculated by using a linear interpolation method, in a case where the corresponding channel ch=−β/dβ is a non-integer, where d β is an interval between channels in detectors. In other words, dβ is the fan-beam angular sampling interval per detector channel.

In some embodiments, the multi-source CT system comprises more than 1 combination of tube and detectors; in a dual-source CT system, an included angle between two combinations of tube and detectors is about 90 degrees; or in a triple-source CT system, an included angle between two adjacent combinations among three combinations of tube and detectors is about 120 degrees.

In some embodiments, determining projection data deviating from the central channel by −β in a projection with a sampling angle of α+180°−2β, as the data corresponding to the bad channel, in a case where performing compensation by using data of a next adjacent detector in the same direction as a gantry's rotation direction, where α is a sampling angle of a certain combination of tube and detectors, and β is the angle by which the bad channel deviate from a central channel; or determining projection data deviating from the central channel by −β in a projection with a sampling angle of α−180°+2β, as the data corresponding to the bad channel, in a case where performing compensation by using data of a previous adjacent detector in the direction as the gantry's rotation direction.

In some embodiments, in the dual-source system:
an angle interval by which the tubes are rotated is (180°−90°−2β), and a time interval is (180°−90°−2β)/360°·r, where r is a rotation speed of the gantry, in a case where the data of the current combination is corrected by using the data of the next adjacent combination of tube and detectors in the rotation direction, where the included angle A between the two combinations of tube and detectors is approximately 90 degrees for one latter combination of detectors in the rotation direction or 270 degrees for one former combination of detectors in the rotation direction, for example, when the rotation speed r is 1 second/rotation and a fan angle Fan of a ray beam formed by all the detectors channels and the ray source is 52 degrees, a value range of β of [−Fan/2, Fan/2] is [−26°, 26° ], and therefore, a time difference for correcting the current data by using the data of the adjacent combination of tube and detectors being between [0.11 r, 0.39 r], which is significantly lower than a time difference of 0.64 r for correcting the current data by using the own combination tube and detectors; or
a time difference same in magnitude but negative as the time difference range while the data of the current combination is corrected by using the data of the next adjacent combination of tube and detectors, in a case where the data of the current combination is corrected by using the data of the previous adjacent combination of tube and detectors, i.e., a time when the corresponding data of the previous combination of tube and detectors is generated is earlier,
wherein exposure and data acquisition of the latter combination are stopped, after the latter combination in the rotation direction passes through the included angle A between the two combinations of tube and detectors, in a case where it is ensured that no repeated scan is performed by the two combinations of tube and detectors, which results in that only the data within the range of the included angle A is capable to be corrected by using the data of the adjacent combination of tube and detectors.

In some embodiments, in the triple-source system: an angle interval by which the tubes are rotated is (180°−120°−2β), and a time interval is (180°−120°−2β)/360°·r, in a case where the data of the current combination is corrected by using the data of the next adjacent combination of tube and detectors in the rotation direction, where the included angle A is approximately 120 degrees, when the rotation speed r is 1 second/rotation and a fan angle Fan is 52 degrees, a value range of βof [−Fan/2, Fan/2] is [−26°, 26°], and a time difference for correcting the current data by using the data of the adjacent combination of tube and detectors being between [0.02 r, 0.31 r], which is significantly lower than a maximum time difference of 0.64 r for a single-source system which can correct the current data only by the own combination of tube and detectors, and a maximum time difference of 0.39 r for dual-source system; or
a time difference same in magnitude but negative as the time difference range while the data of the current combination is corrected by using the data of the next adjacent combination of tube and detectors, in a case where the data of the current combination is corrected by using the data of the previous adjacent combination of tube and detectors, i.e., a time when the corresponding data of the previous combination of tube and detectors is generated is earlier;
wherein data of a previous or next combination of tube and detectors can be found to correct bad data appearing in bad channel of a current combination of tube and detectors, for the triple-source system, since the three combinations of tube and detectors are respectively rotated by 120 degrees for coverage of all scan angles without repeating, for data at all angles;
a value range of α is [0, 120), a possible value range of α projection angle α+180°−2β where an opposite ray is located is [180°−2β, 300°−2β), when a starting angle s is 0, and when β<60°, the angle does not fall within a obtainable range of data angle [120°, 240°) by the next combination of tube and detectors, but falls within an angle range [240°, 360°) of the previous combination of tube and detectors, and the compensation method using the next combination of tube and detectors adopts an angle interval of 240 degrees, or the compensation method using the previous combination of tube and detectors adopts an angle interval of 120 degrees, the two compensation methods having both consistent angle interval results −2β−60° and consistent time interval results r (−60°−2β)/(360°); and
α in the above description is replaced with an angle by which the combination of tube and detectors is rotated from the starting angle, when sampling is started and the starting angle s of the tube is not 0 degree, that is, a in the above description is replaced with α− s, i.e., the algorithm is only related to the angle by which the combination of tube and detectors is rotated relative to the starting angle.

According to another aspect of the present disclosure, there is provided a multi-source computed tomography (CT) system configured to perform the data correction method as described above or the data correction method for bad channel as described above.

The present disclosure is used for ensuring the image quality, improving the yield of detectors, and reducing the system costs.

### BRIEF DESCRIPTION OF THE DRAWINGS

FIGS. 1 and 2 are schematic diagrams of an application of the present disclosure in a dual-source system;

FIGS. 3 and 4 are schematic diagrams of an application of the present disclosure in a triple-source system;

FIG. 5 is a flow diagram of generation and storage of a bad channel distribution map when the present disclosure is applied in a triple-source system;

FIG. 6A is a flow diagram of a data correction method according to some embodiments of the present disclosure;

FIG. 6B is a flow diagram of bad channel correction during a scan when the present disclosure is applied in a triple-source system;

FIG. 7 is a schematic diagram of correcting a current channel by using opposite projection data passing through the same path as the current channel in the present disclosure;

FIG. 8 is a schematic diagram of time differences for correction by using an opposite projection data when the present disclosure is applied in a triple-source system.

### DETAILED DESCRIPTION

The technical solutions in the embodiments of the present disclosure will be clearly and completely described below in conjunction with the drawings in the embodiments of the present disclosure, and it is obvious that the described embodiments are only some of the embodiments of the present disclosure, rather than all of them. All other embodiments, which are obtained by one of ordinary skill in the art based on the embodiments in the present disclosure without making any creative labor, shall fall within the scope of protection of the present disclosure.

In some embodiments, in a data correction method for a bad channel of a multi-source CT system, the multi-source CT system comprises more than 1 combination of tube and detectors. In a dual-source CT system, an included angle between two combinations of tube and detectors is about 90 degrees. In a triple-source CT system, an included angle between two adjacent combinations among three combinations of tube and detectors is about 120 degrees.

A position and distribution of a bad channel can be determined by a linearity check, through acquiring data of different tube currents in the system for multiple times. A projection with the bad channel is a first projection of a first combination of tube and detectors.

The projection data of the bad channel can be corrected by interpolating projection data of adjacent channels of the bad channel in a current detectors, in a case where the distribution of the bad channel meets:
a) adjacent channels each being a good channel;
b) the bad channel being not located within a certain view range (e.g. 20 cm); and
c) the bad channel being not located at an outermost row in a multi-detector acquisition mode supported by the CT system.

In a case where the distribution of the bad channel does not meet the above condition, it is checked, according to the following solution, whether a previous combination of tube and detectors or next combination of tube and detectors is capable of providing opposite projection data to correct the data of the target bad channel. The combination of tube and detectors capable of providing the data for correcting the bad channel is a second combination of tube and detectors.

If the current bad channel can be corrected by using the opposite projection data of the previous combination of tube and detectors or next combination of tube and detectors, the opposite projection data can be selected according to the number of combinations of tube and detectors of the CT system and relative position between combinations of tube and detectors, and then correcting the data, and entering a subsequent pre-processing process.

By the position and distribution of the bad channel, a sampling angle of the first projection and a projection angle of the bad channel in the first projection may be determined, the projection angle being an angle by which the bad channel in the first combination of tube and detectors deviates from a central channel.

For a certain tube and detectors combination, if the bad channel is located deviating from the central channel by β (detectors row with respect to a center), in a projection with an angle α, then:
data corresponding to the bad channel can be determined as projection data deviating from the central channel by −β in a projection with a sampling angle of α+180°−2β, in a case where performing compensation by using data of a next adjacent detector in the same direction as a gantry's rotation direction, where a is a sampling angle of a certain combination of tube and detectors, and β is the angle by which the bad channel deviate from a central channel, resulting in an angle by which the combination of tube and detectors for the compensation rotates is α+180°−2β−(α+A)=180°−A−2β, where angle A is an included angle between the two adjacent combinations of tube and detectors. In the above case, the second projection is obtained after the first projection.

Data corresponding to the bad channel can be determined as projection data deviating from the central channel by −β in a projection with a sampling angle of α−180°+2β, in a case where performing compensation by using data of a previous adjacent detector in the direction as the gantry's rotation direction, resulting in an angle by which the combination of tube and detectors for the compensation is rotated is α−180°−2β−(α−A)=−2β+A−180°, where angle A is an included angle between the two adjacent combinations of tube and detectors In the above case, the second projection is obtained before the first projection.

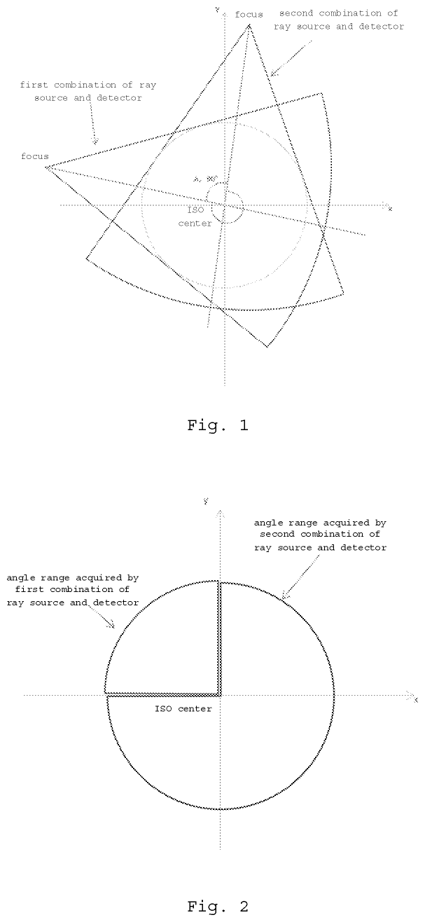

In the dual-source system, the included angle A is approximately 90 and 270 degrees, respectively, as shown in FIG. 1. Therefore, if the data of the current combination is corrected by using the data of the next adjacent combination of tube and detectors in the rotation direction, an angle interval by which the tubes are rotated is (180°−90°−2β), and a time interval is (180°−90°−2β)/360°·r. When the rotation speed (time per rotation of the gantry) r is 1 second/rotation and a fan angle Fan is 52 degrees, a value range of β is [−26°, 26′ ], and therefore, a time difference of correcting the current data by using the data of the adjacent combination of tube and detectors is between [0.11 r, 0.39 r], which is significantly lower than a time interval between projections for data correction in a single-source CT system.

Similarly, a time difference range same in magnitude but negative, as the time difference range while the data of the current combination is corrected by using the data of the next adjacent combination of tube and detectors, can be determined, in a case where the data of the current combination is corrected by using the data of the previous adjacent combination of tube and detectors, i.e., a time when the corresponding data of the previous combination of tube and detectors is generated is earlier.

If it is ensured that no repeated scan is performed by the two combinations of tube and detectors, after the latter combination in the rotation direction passes through the included angle A between the two combinations of tube and detectors, exposure and data acquisition of the latter combination should be stopped (as shown in FIG. 2), which results in that only the data within the range of the included angle A is capable to be corrected by using the data of the adjacent combination of tube and detectors.

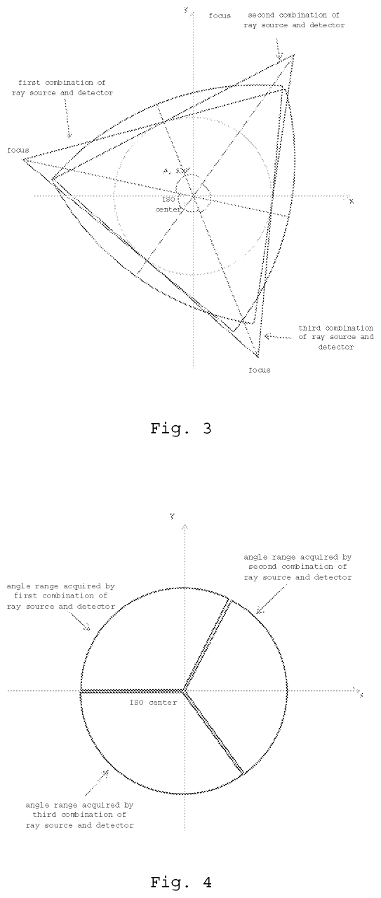

In the triple-source system, the included angle A is approximately 120 degrees, respectively, as shown in FIG. 3. Therefore, if the data of the current combination is corrected by using the data of the next adjacent combination of tube and detectors in the rotation direction, an angle interval by which the tubes are rotated is (180°−120°−2β), and a time interval is (180°−120°−2β)/360°·r. When the rotation speed r is 1 second/rotation and a fan angle Fan is 52 degrees, a value range of β is [−26°, 26°], and therefore, a time difference of correcting the current data by using the data of the adjacent combination of tube and detectors is between [0.02 r, 0.31 r], which is significantly lower than the time interval between the projections for data correction in the single-source CT system and the time interval between the projections for data correction in the dual-source CT system.

Similarly, a time difference range same in magnitude but negative, as the time difference range while the data of the current combination is corrected by using the data of the next adjacent combination of tube and detectors, can be determined, in a case where the data of the current combination is corrected by using the data of the previous adjacent combination of tube and detectors, i.e., a time when the corresponding data of the previous combination of tube and detectors is generated is earlier.

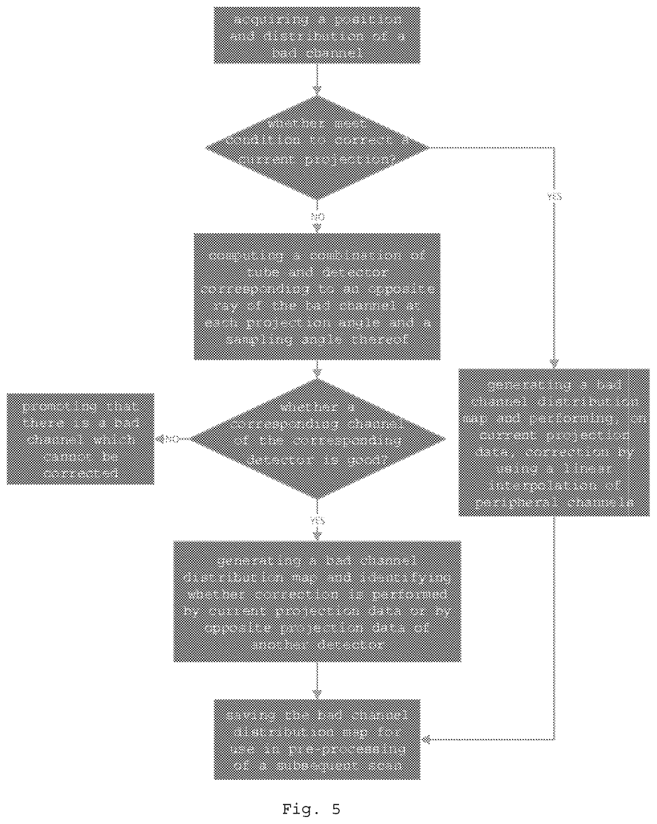

For the triple-source system, since the three combinations of tube and detectors are respectively rotated by 120 degrees without repeated coverage of all scan angles, as shown in FIG. 4, for data at all the angles, data of a previous or next combination of tube and detectors can be found to correct bad data appearing in a bad channel of current combination of tube and detectors.

When a starting angle s is 0, a value range of α is [0, 120). A possible value range of an angle α+180°−2β of a projection where an opposite ray is located is [180°−2β, 300°−2β). When β<60°, the angle does not fall within a range of data angle [120°, 240°) which is obtainable by the next combination of tube and detectors, but falls within an angle range of [240°, 360°) of the previous combination (which can also be considered as a 2nd latter combination with an interval of 240 degrees). Therefore, it is possible to use the next combination of tube and detectors for the compensation method with use of an angle interval of 240 degrees or use the previous combination of tube and detectors for the compensation method with use of an angle interval of 120 degrees, the two having consistent angle interval −2β−60° and consistent time interval results

$$r \frac{- 60 ° - 2 β}{360 °} .$$

When the starting angle s is not 0 degree, α in the above description may be replaced with an angle by which the combination of tube and detectors is rotated from the starting angle. That is, α in the above description is replaced with α−s. The algorithm is only related to the angle by which the combination of tube and detectors is rotated relative to the starting angle. It should be understood that when the starting angle s is not 0 degree, a value range of α is [0+s, 120+s), and a possible value range of the sampling angle α+180°−2β for the projection where the opposite ray is located is [180°+s−2β, 300°+s−2β].

After the corresponding combination of tube and detectors and the sampling angle are determined, according to an angle between the channel and the central channel, a corresponding channel is calculated. If a channel interval is dβ degrees, the corresponding channel is ch=−β/dβ. If ch is an integer, it is indicated that the corresponding channel exists, and projection data of the channel ch is directly used as the opposite projection data. And if ch is a non-integer, a corresponding projection value may be calculated by using a linear interpolation method.

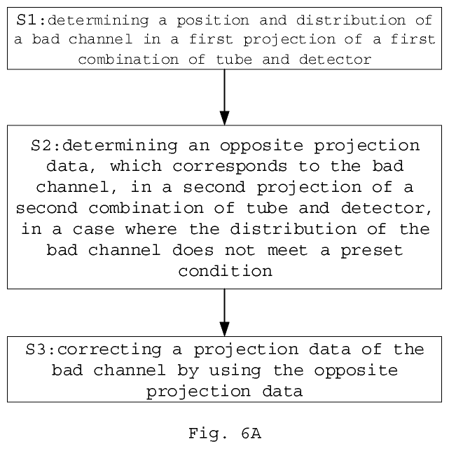

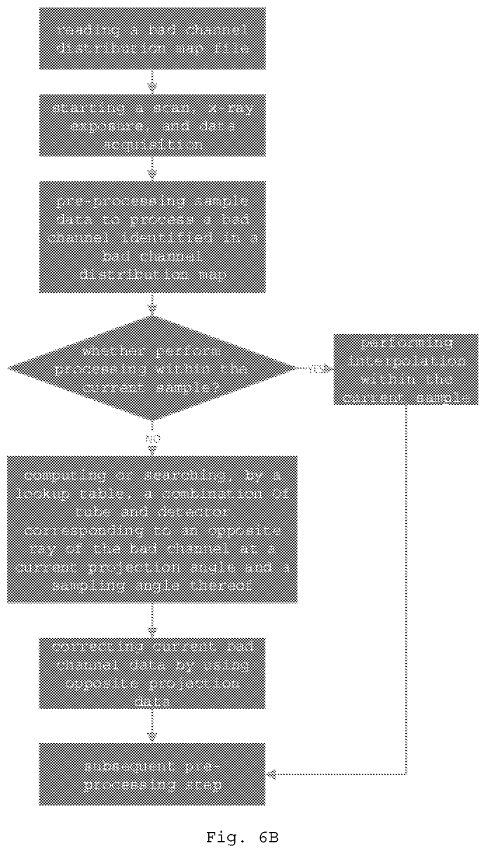

In the CT system, the process of the data correction includes two parts: generation and storage of a bad channel distribution map (FIG. 5), and bad channel correction during scan (FIGS. 6A-6B).

Next, a process of a data correction method according to some embodiments of the present disclosure will be described with reference to FIG. 6A. As shown in FIG. 6A, the data correction method includes: step S1, determining a position and distribution of a bad channel in a first projection of a first combination of tube and detectors; step S2, determining an opposite projection data, which corresponds to the bad channel, in a second projection of a second combination of tube and detectors, in a case where the distribution of the bad channel does not meet a preset condition; and step S3, correcting a projection data of the bad channel by using the opposite projection data.

Since the opposite projection data for the triple-source CT system is more complete than that for the dual-source system, and the single-source system is relatively simple, only the process of correcting the bad channel data by using the opposite projection data in the triple-source system is given here, as shown in FIG. 6B.

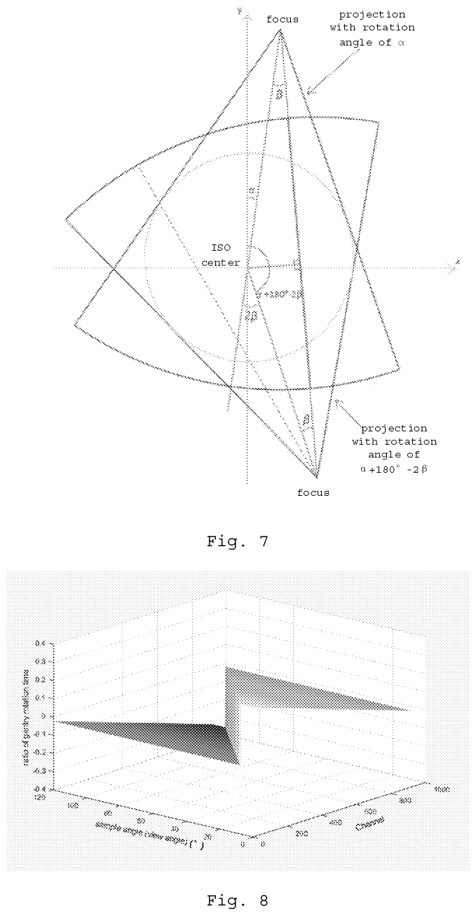

To reduce or relax the requirements for the distribution of the bad channel, the current channel may be corrected by using opposite projection data passing through the same path. As shown in FIG. 7, for a projection that deviates from a central channel by an angle β, in a fan projection with an angle α, its projection line is same as a projection line that deviates from the central channel by an angle −β, in a projection with an angle α+180°−2 β, and therefore can be used for compensation for each other. (where, β is an angle for which a focus is taken as a center and a straight line passing through the central channel is taken as 0 degree. The preceding sign “−” indicates an opposite direction to β. Assuming that a fan angle is Fan, a value range of β is [−Fan/2, +Fan/2].)

For a CT system with a wider detectors, due to the effect of a cone angle, for an outer row of the detectors in a z-direction, it is not suitable to perform compensation in this way, but errors for several rows close to a center of the detectors in the z-direction are small enough.

For the compensation for each other by using the data of the two projections with α and α+180°−2β, although it brings more data for compensation, there still are limitations, since there are various movements of a patient himself in medical CT, such as breathing, heartbeat, peristalsis of gastrointestinal tract in his body, etc.

In the single-source CT system, for α and α+180°−2β, a maximum interval between the two is 1800+Fan due to the value range of β of [−Fan/2, +Fan/2]. Assuming that a gantry rotation speed is r seconds/rotation, a time interval between the two projections is r (180°+Fan)/360. The interval is 0.64 r when the fan angle is 52 degrees. Therefore, the time interval is relatively large, and compensation by using data with a relatively large time interval is vulnerable to the movement of the patient to generate artifacts.

In the dual-source system of the present disclosure, the current data is corrected by using data from the adjacent combination of tube and detectors, and when the fan angle is 52 degrees, the interval is between [0.11 r, 0.39 r], which is significantly lower than the maximum 0.64 r of the time interval between the current data and correction data when correction is performed by using the own combination of tube and detectors.

In the triple-source system of the present disclosure, the current data is corrected by using data from the adjacent combination of tube and detectors, and when the fan angle is 52 degrees, the interval is between [0.02 r, 0.31 r], which is much significantly lower than the maximum 0.64 r of the time interval between the current data and correction data when correction is performed by using the own combination of tube and detectors, and is also lower than the maximum 0.39 r of the time interval in the dual-source system.

For the triple-source system, when calculation is performed according to the interval as 120 degrees and the fan angle of 52 degrees, at sampling angles for channels, time differences for correction by using an opposite projection data are shown in FIG. 8. It can be seen that a maximum time difference is 0.31 time the gantry's rotation speed.

By the data correction method provided in the present disclosure, a time interval between a projection for correction and a projection with a bad channel is significantly reduced, thereby ensuring the image quality, improving the yield of detectors and reducing the system costs.

In some embodiments, the present disclosure further provides a multi-source computed tomography (CT) system capable of performing the data correction method as described above or the data correction method for bad channel as described above, thereby ensuring the image quality, improving the yield of detectors, and reducing the system costs.

So far, various embodiments of the present disclosure have been described in detail. Some details well known in the art have not been described in order to avoid obscuring the concepts of the present disclosure. Those skilled in the art can fully appreciate how to implement the technical solution disclosed herein according to the foregoing description.

Although some specific embodiments of the present disclosure have been described in detail by examples, it should be understood by those skilled in the art that the above examples are for illustration only and are not intended to limit the scope of the present disclosure. It should be understood by those skilled in the art that the above embodiments may be modified and part of the technical features may be equivalently substituted without departing from the scope and spirit of the present disclosure. The scope of the present disclosure is defined by the appended claims.

1. A data correction method for a multi-source computed tomography (CT) system, comprising:
determining a position and distribution of a bad channel in a first projection of a first combination of tube and detectors;
determining an opposite projection data, which corresponds to a channel opposite to the bad channel, in a second projection of a second combination of tube and detectors, in a case where the distribution of the bad channel does not meet a preset condition; and
correcting a projection data of the bad channel by using the opposite projection data.

2. The data correction method according to claim 1, wherein the determining an opposing projection data in the second projection of the second combination of tube and detectors corresponding to the bad channel, comprises:
determining a sampling angle of the first projection and a projection angle of the bad channel according to the position and distribution of the bad channel;
determining the second projection according to the sampling angle of the first projection and the projection angle of the bad channel;
determining an opposite projection angle in the second projection corresponding to the bad channel according to the projection angle of the bad channel; and
determining the opposite projection data according to the opposite projection angle.

3. The data correction method according to claim 2, wherein the determining the second projection according to the sampling angle of the first projection and the distribution of the bad channel, comprises:
determining a projection of the second combination of tube and detectors at a sampling angleα+180°−2β as the second projection, in a case where the second projection is acquired after the first projection, wherein α is the sampling angle of the first projection, and β is the projection angle of the bad channel, the projection angle being an angle by which the bad channel in the first combination of tube and detectors deviates from a central channel; or
determining a projection of the second combination of tube and detectors at a sampling angle α−180°+2β as the second projection, in a case where the second projection is acquired before the first projection.

4. The data correction method according to claim 3, wherein the opposite projection angle is an angle of −β, by which a channel in the second combination of tube and detectors corresponding to the bad channel deviates from the central channel.

5. The data correction method according to claim 4, wherein the determining the opposite projection data according to the opposite projection angle, comprises:
determining that a channel in the second combination of tube and detectors combination corresponding to the opposite projection angle is ch=−β/dβ, according to the opposite projection angle −β and a channel interval dβ in the second combination of tube and detectors;
determining the opposite projection data according to projection data of channel ch, in a case where ch is an integer; and
calculating projection data corresponding to the channel ch according to projection data of adjacent channels by using a linear interpolation method, in a case where ch is a non-integer.

6. The data correction method according to claim 1, further comprising:
determining an angle by which a gantry of the CT system is rotated between the first projection and the second projection, according to the first projection of the first combination of tube and detectors and the second projection of the second combination of tube and detectors.

7. The data correction method according to claim 6, wherein the determining an angle by which a gantry of the CT system is rotated between the first projection and the second projection, comprises:
determining that the angle by which the gantry is rotated is α+180°−2β−(α+A)=180°−A−2β, in a case where the second projection is the projection of the second combination of tube and detectors at a sampling angle of α+180°−2β, wherein angle A is an included angle between the first combination of tube and detectors and the second combination of tube and detectors; and/or
determining that the angle by which the gantry is rotated is α−180°+2β−(α−A)=2β+A−180°, in a case where the second projection is the projection of the second combination of tube and detectors at the sampling angle of α−180°+2β.

8. The data correction method according to claim 7, wherein the CT system is a dual-source CT system, the included angle A between the first combination of tube and detectors and the second combination of tube and detectors is 90° or 270°, and the angle by which the gantry is rotated is 180°−90°−2β or 2β+90°−180°.

9. The data correction method according to claim 7, wherein the CT system is a triple-source CT system, the included angle A between the first combination of tube and detectors and the second combination of tube and detectors is 120°, and the angle by which the gantry is rotated is 180°−120°−2β or 2β+120°−180°.

10. The data correction method according to claim 1, wherein the CT system is a triple-source CT system, in a case where the distribution of the bad channel does not meet the preset condition, the method further comprises:
determining the second combination of tube and detectors according to a starting angle s of the triple-source CT system, a sampling angle α for the first projection, and a projection angle β of the bad channel.

11. The data correction method according to claim 10, wherein the determining the second combination of tube and detectors, comprises:
determining that the second combination of tube and detectors is first in a clockwise direction with respect to the first combination of tube and detectors, in a case where a value of α+180°−2β is within an angle range of [120°+s,240°+s); and
determining that the second combination of tube and detectors is first in a counter clockwise direction with respect to the first combination of tube and detectors, in a case where a value of α+180°−2β is within an angle range of [240°+s,360°+s).

12. The data correction method according to claim 1, further comprising:
interpolating projection data of adjacent channels of the bad channel in the first projection, to correct the projection data of the bad channel, in a case where the distribution of the bad channel meets the preset condition, wherein the preset condition comprises:
the adjacent channels of the bad channel each being a good channel;
the bad channel being not located within a certain view range close to a central channel; and
the bad channel being not located at an outermost row in a multi-detector acquisition mode supported by the CT system.

13. A data correction method for a bad channel of a multi-source CT system, comprising:
determining a position and distribution of a bad channel by a linearity check, through acquiring data of different tube currents in the system for multiple times;
interpolating projection data of adjacent channels of the bad channel in a current detectors, to correct the projection data of the bad channel, in a case where the distribution of the bad channel meets conditions of adjacent channels each being a good channel the bad channel being not located within a certain view range close to a central channel and the bad channel being not located at an outermost row in a multi-detector acquisition mode supported by the CT system;
checking whether a previous combination of tube and detectors or next combination of tube and detectors is capable of providing opposite projection data, which corresponds to a channel opposite to the bad channel, to correct the data of a target bad channel, in a case where the distribution of the bad channel does not meet the conditions; and
selecting the opposite projection data according to a number of combinations of tube and detectors of the CT system and relative position between combinations of tube and detectors, and then correcting the data of the bad channel with the opposite projection data, in a case where it is capable to correct the target bad channel by using the opposite projection data of the previous combination of tube and detectors or the next combination of tube and detectors,
wherein the selecting the opposite projection data according to the number of combinations of tube and detectors of the CT system and relative position between combinations of tube and detectors, specifically comprises:
determining projection data deviating from the central channel by −β in a projection with a sampling angle of α+180°−2β, as the data corresponding to the bad channel, in a case where performing compensation by using data of a next adjacent detector in a same direction as a gantry's rotation direction, where α is a sampling angle of a combination of tube and detectors with the bad channel, and β is the angle by which the bad channel deviate from a central channel, resulting in an angle by which the combination of tube and detectors for the compensation rotates is α+180°−2β−(α+A)=180°−A−2β, where angle A is an included angle between the two adjacent combinations of tube and detectors; or
determining projection data deviating from the central channel by −β in a projection with a sampling angle of α−180°+2β, as the data corresponding to the bad channel, in a case where performing compensation by using data of a previous adjacent detector in the same direction as the gantry's rotation direction, resulting in an angle by which the combination of tube and detectors for the compensation is rotated is α−180°−2β−(α−A)=−20+A−180°, where angle A is an included angle between the two adjacent combinations of tube and detectors,
wherein determining projection data comprises calculating a corresponding channel according to the included angle between the target channel and the central channel, after determining a corresponding combination of tube and detectors and sampling angle, and calculating a corresponding projection value by using a linear interpolation method, in a case where the corresponding channel ch=−β/dβ is a non-integer, where dβ is an interval between channels in detectors.

14. The data correction method for a bad channel of a multi-source CT system according to claim 13, wherein the multi-source CT system comprises more than one combination of tube and detectors, in which:
in a dual-source CT system, an included angle between two combinations of tube and detectors is about 90 degrees; or
in a triple-source CT system, an included angle between two adjacent combinations among three combinations of tube and detectors is about 120 degrees.

15. The data correction method for a bad channel of a multi-source CT system according to claim 14, wherein in the dual-source CT system:
an angle interval by which the tubes are rotated is (180°−90°−2β), and a time interval between the data of the bad channel and the data used for correction is (180°−90°−2β)/360°·r, where r is a rotation speed of the gantry, in a case where the data of the current combination is corrected by using the data of the next adjacent combination of tube and detectors in the rotation direction, where the included angle A between the two combinations of tube and detectors is approximately 90 degrees for one latter combination of detectors in the rotation direction or 270 degrees for one former combination of detectors in the rotation direction; or
a time difference range between the data of the bad channel and the data used for correction is same in magnitude but negative as the time difference range while the data of the current combination is corrected by using the data of the next adjacent combination of tube and detectors, in a case where the data of the current combination is corrected by using the data of the previous adjacent combination of tube and detectors,
wherein exposure and data acquisition of the latter combination are stopped, after the latter combination in the rotation direction passes through the included angle A between the two combinations of tube and detectors, in a case where it is ensured that no repeated scan is performed by the two combinations of tube and detectors, which results in that only the data within the range of the included angle A is capable to be corrected by using the data of the adjacent combination of tube and detectors.

16. The data correction method for a bad channel of a multi-source CT system according to claim 14, wherein in the triple-source CT system:
an angle interval by which the tubes are rotated is (180°−120°−2β), and a time interval between the data of the bad channel and the data used for correction is (180°−120°−2β)/360°·r, in a case where the data of the current combination is corrected by using the data of the next adjacent combination of tube and detectors in the rotation direction, where the included angle A is approximately 120 degrees; or
a time difference range between the data of the bad channel and the data used for correction same in magnitude but negative as the time difference range while the data of the current combination is corrected by using the data of the next adjacent combination of tube and detectors, in a case where the data of the current combination is corrected by using the data of the previous adjacent combination of tube and detectors,
wherein data of a previous or next combination of tube and detectors can be found to correct bad data appearing in bad channel of a current combination of tube and detectors, for the triple-source system, since the three combinations of tube and detectors are respectively rotated by 120 degrees for coverage of all scan angles without repeating, for data at all angles;
a value range of α is [0, 120), a possible value range of α projection angle α+180°−2β where an opposite ray is located is [180°−20, 300°−2β), when a starting angles is 0, and when β<60°, the angle does not fall within a obtainable range of data angle [120°, 240°) by the next combination of tube and detectors, but falls within an angle range [240°, 360°) of the previous combination of tube and detectors, and the compensation method using the next combination of tube and detectors adopts an angle interval of 240 degrees, or the compensation method using the previous combination of tube and detectors adopts an angle interval of 120 degrees, the two compensation methods having both consistent angle interval results −2β−60° and consistent time interval results
$$r \frac{- 60 ° - 2 β}{360 °} ;$$
and
wherein α is replaced with an angle by which the combination of tube and detectors is rotated from the starting angle, when sampling is started and the starting angle s of the tube is not 0 degree.

17. A multi-source computed tomography (CT) system configured to perform a data correction method, comprising:
determining a position and distribution of a bad channel in a first projection of a first combination of tube and detectors:
determining an opposite projection data, which corresponds to a channel opposite to the bad channel, in a second projection of a second combination of tube and detectors, in a case where the distribution of the bad channel does not meet a preset condition; and
correcting a projection data of the bad channel by using the opposite projection data.

18. A multi-source computed tomography (CT) system configured to perform the data correction method according to claim 13.

19. The data correction method for a bad channel of a multi-source CT system according to claim 15, wherein the rotation speed r is 1 second/rotation, a fan angle Fan of a ray beam formed by all the detectors channels and the tube is 52 degrees, and determining a time interval comprises:
determining a value range of β of [−Fan/2, Fan/2] is [−26°, 26° ]; and
determining a time difference for correcting the current data by using the data of the adjacent combination of tube and detectors is between [0.1 r, 0.39 r], which is significantly lower than a time difference of 0.64 r for correcting the current data in a single-source system.

20. The data correction method for a bad channel of a multi-source CT system according to claim 16, wherein a rotation speed r is 1 second/rotation, a fan angle Fan is 52 degrees, and determining a time interval comprises:
determining a value range of β of [−Fan/2, Fan/2] is [−26°, 26° ]; and
determining a time difference for correcting the current data by using the data of the adjacent combination of tube and detectors being between [0.02 r, 0.31 r], which is significantly lower than a maximum time difference of 0.64 r for a single-source system, and a maximum time difference of 0.39 r for dual-source system.
